(19) United States
(12) Patent Application Publication
(10) Pub. No.: US 2025/0161448 A1
(43) Pub. Date: May 22, 2025

(54) GD2 CHIMERIC ANTIGEN RECEPTOR AND USE THEREOF

(71) Applicant: BEIJING MEIKANG GENO-IMMUNE BIOTECHNOLOGY CO., LTD., Beijing (CN)

(72) Inventor: Yuchen Liu, Beijing (CN)

(73) Assignee: BEIJING MEIKANG GENO-IMMUNE BIOTECHNOLOGY CO., LTD., Beijing (CN)

(21) Appl. No.: 18/843,101

(22) PCT Filed: Mar. 1, 2023
(86) PCT No.: PCT/CN2023/079018

§ 371 Date: Aug. 30, 2024

(30) Foreign Application Priority Data
Mar. 2, 2022 (CN) 202210197703.2

(51) Int. Cl. A61K 40/31; A61K 35/17; A61K 40/11; A61K 40/42; A61P 35/00; C07K 14/705; C07K 14/715; C07K 14/725; C07K 16/30; C12N 5/0783; C12N 9/64; C12N 15/86

(52) U.S. Cl. CPC A61K 40/31; A61K 35/17; A61K 40/11; A61K 40/4202; A61P 35/00; C07K 14/7051; C07K 14/70517; C07K 14/70521; C07K 14/7155; C07K 16/3084; C12N 5/0636; C12N 9/6472; C12N 15/86; A61K 2239/13; A61K 2239/21; A61K 2239/25; A61K 2239/47; C07K 2317/24; C07K 2317/622; C07K 2319/03; C07K 2319/33; C12N 2510/00; C12N 2740/15043; C12Y 304/22062

(57) ABSTRACT

Provided are a disialoganglioside 2 (GD2) chimeric antigen receptor (CAR) and use thereof. A humanized GD2 single-chain variable fragment (scFv) antibody has activity of binding to a GD2 antigen, where the humanized GD2 scFv has a more than 80% of amino acid sequence identity with SEQ ID NO. 1. Further provided are the GD2 CAR and a chimeric antigen receptor T (CAR-T) cell expressing the GD2 CAR. The humanized GD2 scFv has better bioactivity and compatibility. Binding the GD2 CAR to GD2 has a better response effect, a stronger immune response and a better long-term effect. The CAR-T cell has higher safety and persistence and an extremely high application value.

### TECHNICAL FIELD

The present application belongs to the technical field of tumor immunotherapies and, in particular, relates to a disialoganglioside 2 (GD2) chimeric antigen receptor (CAR) and use thereof.

### BACKGROUND

With the development of tumor immunology theories and clinical techniques, chimeric antigen receptor T (CAR-T) cell immunotherapy has become one of the most promising tumor immunotherapies. In general, a CAR consists of a tumor-associated antigen (TAA) binding region, an extracellular hinge region, a transmembrane region and an intracellular signaling region. Generally, the CAR contains a single-chain variable fragment (scFv) region of an antibody or a domain specifically binding to a TAA, which is coupled to a cytoplasmic domain of a T-cell signaling molecule through hinge and transmembrane regions. The most common lymphocyte activating moiety includes a T-cell costimulatory domain in tandem with a moiety (for example, CD3ζ) triggering the function of a T-cell effector.

CAR-mediated adoptive immunotherapy allows CAR-modified T cells to directly recognize TAAs on target tumor cells in a non-human leukocyte antigen (HLA)-restricted manner. At present, the treatment of CAR-T achieves an exciting effect on hematological tumors, and it is proved that a second generation CD19 CAR-T has an anti-tumor efficacy on recurrent and refractory B acute lymphocytic leukemia, chronic lymphocytic leukemia and lymphoma with an overall response rate of about 50% to 90% according to different tumors.

Disialoganglioside 2 (GD2), which is widely expressed in tumors such as neuroblastoma, melanoma, glioma and sarcoma and is expressed in a low amount and limitedly in normal tissues, is an ideal tumor antigen for immunotherapies. At present, this cancer antigen is mostly used in the treatment of neuroblastoma, which is the most common extracranial solid malignant tumor in children. About 50% of the children with neuroblastoma have large-scale spread and metastasis of the tumor after the onset of the disease. Conventional surgery, chemotherapy, radiotherapy and autologous stem cell transplantation have limited therapeutic effects on this group of patients, and even after the disease is controlled to be in remission, more than 80% of the patients suffer from the recurrence of the disease and die within one year.

At present, among the immunotherapies of neuroblastoma, a relatively mature immunotherapy is antibody treatment for GD2, which achieves a preliminary clinical success. However, a published clinical research report indicates that GD2 monoclonal antibody therapy has relatively high toxicity, 52% of the patients have grades 3 to 4 toxicity reactions and some patients have neurotoxicity. In addition, the antibody is mainly present in peripheral blood after administration. It is relatively difficult for the antibody to accurately enter tumor tissues or sites where a minor tumor remains, and the antibody cannot be present in vivo for a long time. Therefore, in addition to advantages of antibody treatment, the prepared CAR GD2 CAR-T cell can accurately enter the tumor tissues and be present in vivo for a long time due to characteristics of the T cell, which can provide a more effective treatment option for children with recurrent and refractory neuroblastoma. At present, clinical reports of the treatment of neuroblastoma through GD2 CAR-T have shown a preliminary efficacy of CAR-T. However, there is still a lack of long-term observation data.

At present, an effect of CAR-T technology in treating solid tumors is not very ideal. Many scFv regions of CAR-T are derived from murine antibodies, and the murine ScFv is easily rejected by a human immune system, which causes that CAR-T cannot be present in vivo for a long time, thereby limiting a therapeutic effect. It has been reported that this is also one of the reasons of recurrence in many patients with acute lymphocytic leukemia after complete remission by using CD19 CAR-T and also causes the difficulty of retreatment.

Therefore, how to provide a humanized GD2 CAR-T with a good therapeutic effect and a long duration has become an urgent problem to be solved.

### SUMMARY

The present application provides a GD2 CAR and use thereof. CAR-T containing the GD2 CAR can remove GD2-positive solid tumors, effectively remove minor residues in bone marrow without adverse reactions and improve the safety, efficacy, memory and long-term maintenance of CAR-T in combination with an optimized T cell signaling region.

In a first aspect, the present application provides a humanized GD2 scFv. The humanized GD2 scFv has activity of binding to a GD2 antigen.

The humanized GD2 scFv has an amino acid sequence having more than 80% identity with SEQ ID NO. 1.

SEQ ID NO. 1:
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSGSTSGSGKPGSSEGSTKGDIVMSQSPSSLAV
SVGEKVTMSCKASQSVSNDVTWYQQKPGQSPKLLIYSASNRYSGVPDRFT
GSGSGTDFTLTISSVKAEDLAVYYCQQDYSSFGAGTKLELK.

In the present application, the humanized GD2 scFv has been subjected to specific modification regarding humanization and structure against the antigen GD2 on the surface of tumors. The modified scFv antibody has a stronger function and better compatibility in a human body and is not easily rejected by an immune system.

In a second aspect, the present application provides a derivative antibody conjugate of the humanized GD2 scFv according to the first aspect.

In a third aspect, the present application provides a nucleic acid molecule. The nucleic acid molecule encodes the humanized GD2 scFv according to the first aspect or the derivative antibody conjugate of the humanized GD2 scFv according to the second aspect.

Preferably, the nucleic acid molecule has a nucleotide sequence having more than 80% identity with SEQ ID NO. 2.

SEQ ID NO. 2:
caggtccagttggtcgaatctgggcctggggttgtacagccaggtcgaag
tcttcgcatatcttgtgcagtatctggcttcagtgttaccaattacggtg
tacactgggtgagacaacccccaggtaaagggttggaatggcttggagtg
atatgggctgggggcattactaactacaactccgcttttatgtcacggct
gactatttcaaaagataactccaagaatacggtgtacttgcaaatgaatt
cactccgcgcagaggatacagctatgtattattgtgccagtcggggcggc
cattacggctatgctctggactattggggacaagggacgctcgttacggt
atccagtggtagtacgtcaggctccgggaaacctggaagctcagagggta
gcaccaaaggcgatatcgtcatgtcacaatccccaagttccctcgcggta
agcgttggggagaaggtaaccatgtcttgtaaggcatctcagtcagtttc
aaatgacgtgacgtggtaccaacagaagccgggacaatctcctaagttgt
tgatctactctgcaagtaacaggtactccggagtgccggaccgctttacc
ggctctggctcaggcacggacttcacgctgacgatatctagcgtcaaagc
cgaagacctcgcggtgtattactgtcaacaagattactctagtttcggtg
ctggaacgaagctggagctgaaa.

In a fourth aspect, the present application provides a humanized GD2 CAR. The GD2 CAR includes a GD2-antigen-binding scFv domain, a transmembrane domain, a costimulatory signaling region, a CD3ζ signaling domain and an inducible suicide fusion domain.

The GD2 antigen binding scFv domain includes the humanized GD2 scFv according to the first aspect or the derivative antibody conjugate of the humanized GD2 scFv according to the second aspect.

In the present application, a CAR targeting GD2 is provided that comprises a costimulatory signaling region, a CD3ζ signaling domain and an inducible caspase 9 (iCasp9) suicide fusion domain in tandem, as well as a humanized antigen binding domain that binds to antigen GD2 on tumor surface and has been subjected to specific gene modification. Compared with other CARs, the CAR has a better binding ability to the antigen, thereby improving the effect of CAR-T.

Preferably, the transmembrane domain includes a CD28 transmembrane domain and/or a CD8α transmembrane domain. In some specific embodiments, the transmembrane domain may be selected or modified through an amino acid substitution.

According to the present application, the costimulatory signaling region is a combination of a CD28 costimulatory signaling domain and a T co-signal signaling domain. The CD28 costimulatory signaling domain and the T co-signal signaling domain may be adjusted as required by those skilled in the art, and the arrangement of the CD28 costimulatory signaling domain and the T co-signal signaling domain does not affect the CAR.

Preferably, the costimulatory signaling region includes CD28 and CD27 costimulatory signaling regions or CD28 and IL-15Ra costimulatory signaling regions.

In a specific embodiment, the CAR according to the present invention includes a CD28 hinge region, a CD28 transmembrane domain, a CD28 costimulatory signaling domain and a CD27 costimulatory signaling domain (abbreviated herein as CD28-CD27). The CD28 costimulatory signaling domain and the CD27 costimulatory signaling domain may be linked through a linker. In a more specific embodiment, the CD28-CD27 has an amino acid sequence having more than 90% of identity with the amino acid sequence as shown in SEQ ID NO. 3. In a more specific embodiment, CD28-CD27 has an amino acid sequence as shown in SEQ ID NO. 3.

SEQ ID NO. 3:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSQRRKYRSNKGESPVEPAEPCHYSCPREEEGS
TIPIQEDYRKPEPACSP.

In another specific embodiment, the CAR according to the present invention includes a CD28 hinge region, a CD28 transmembrane domain, a CD28 costimulatory signaling domain and an IL-15Ra costimulatory signaling domain (abbreviated herein as CD28-IL-15Ra). The CD28 costimulatory signaling domain and the IL-15Ra costimulatory signaling domain may be linked through a linker. In a more specific embodiment, the CD28-IL-15Ra has an amino acid sequence having more than 90% of identity with the amino acid sequence as shown in SEQ ID NO. 4. In a more specific embodiment, CD28-IL-15Ra has an amino acid sequence as shown in SEQ ID NO. 4.

SEQ ID NO. 4:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSKSRQTPPLASVEMEAMEALPVTWGTSSRDED
LENCSHHL.

Preferably, the inducible suicide fusion domain includes a caspase 9 domain fused to an FK506 binding protein (FKBP), abbreviated herein as FKBP.Casp9.

Preferably, the FKBP.Casp9 domain has an amino acid sequence having more than 90% identity with SEQ ID NO. 5.

SEQ ID NO. 5:
MGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLEGGGGSGGGGSGAMVGALESLRGNADLAYILSMEPCGHCLIIN
NVNFCRESGLRTRTGSNIDCEKLRRRFSSLHFMVEVKGDLTAKKMVLALL
ELARQDHGALDCCVVVILSHGCQASHLQFPGAVYGTDGCPVSVEKIVNIF
NGTSCPSLGGKPKLFFIQACGGEQKDHGFEVASTSPEDESPGSNPEPDAT
PFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSA
S.

Preferably, the GD2 CAR further includes a signal peptide and/or a 2A sequence.

According to the present application, the CAR further includes a signal peptide. The signal peptide may be a signal peptide capable of directing the transmembrane transfer of the CAR, and those skilled in the art may select a conventional signal peptide of a secretory protein gene in the art as required.

According to the present application, the inducible suicide fusion domain is in tandem with the CD3ζ signaling domain through the 2A sequence, where the 2A sequence can break a protein expressed by the inducible suicide fusion domain from a protein of the CAR, thereby causing the CAR to function; through the injection of an activator, the inducible suicide fusion domain is activated, thereby causing the CAR to be out of function.

Preferably, the signal peptide includes a Secretory signal peptide.

Preferably, the Secretory signal peptide is a signal peptide of a CD8α gene, and the Secretory signal peptide has an amino acid sequence as shown in SEQ ID NO. 6.

SEQ ID NO. 6:
MALPVTALLLPLALLLHAARP.

Preferably, the Secretory signal peptide is a signal peptide of a GM-CSFR gene, and the Secretory signal peptide has an amino acid sequence as shown in SEQ ID NO. 7.

SEQ ID NO. 7:
MLLLVTSLLLCELPHPAFLLIP.

In the present application, the CAR further includes a hinge region having an amino acid sequence of a combination of multiple GGGGS (SEQ ID NO. 10), which may be, for example, GGGGSGGGGS (SEQ ID NO. 11). The hinge region may be selected by those skilled in the art according to an actual situation, which is not particularly limited here. The presence of the hinge region does not affect the performance of the CAR of the present application.

Preferably, the GD2 CAR includes a Secretory signal peptide, a GD2 antigen binding scFv domain, a transmembrane domain, a costimulatory signaling region, a CD3ζ signaling domain, a 2A sequence and an inducible suicide fusion domain.

As a preferred technical solution, the CAR is formed by a Secretory signal peptide, a GD2 antigen binding scFv domain, a CD8α and/or CD28 transmembrane domain, CD28 and CD27 costimulatory signaling regions, a CD3ζ signaling domain, a 2A sequence and a caspase 9 domain in tandem, and the specific arrangement is as follows: Secretory signal-GD2 scFv-CD28-CD27-CD3ζ-2A-FKBP.Casp9.

Alternatively, the CAR is formed by a Secretory signal peptide, a GD2 antigen binding scFv domain, a CD8α and/or CD28 transmembrane domain, a CD28 and IL-15Ra costimulatory signaling regions, a CD3ζ signaling domain, a 2A sequence and a caspase 9 domain in tandem, and the specific arrangement is as follows: Secretory signal-GD2 scFv-CD28-IL-15Ra-CD3ζ-2A-FKBP.Casp9.

In the present application, the CAR further includes a promoter. The promoter is EF1a or any highly expressed promoter.

In a fifth aspect, the present application provides a nucleic acid molecule. The nucleic acid molecule encodes the GD2 CAR according to the fourth aspect.

In a sixth aspect, the present application provides a viral vector. The viral vector includes at least one copy of the nucleic acid molecule according to the fifth aspect.

In the present application, the viral vector can effectively modify immune cells to prepare targeting cells.

Preferably, the viral vector includes a lentiviral vector or a retroviral vector, preferably the lentiviral vector.

In a seventh aspect, the present application provides a recombinant virus, which is obtained by co-transferring the viral vector according to the sixth aspect and a packaging helper plasmid into a mammalian cell.

Preferably, the packaging helper plasmid includes pNHP and pHEF-VSVG.

Preferably, the mammalian cell includes any one of a 293 cell, a 293T cell or a TE671 cell.

In an eighth aspect, the present application provides a CAR-T cell. The CAR-T cell expresses the GD2 CAR according to the fourth aspect.

In the present application, the CAR-T cell has a good targeting and killing effect while releasing a low level of immune factor, showing response properties with low toxicity and high immune killing. The schematic mechanism diagram of the CAR-T cell is shown in FIG. 1.

Preferably, the CAR-T cell is prepared through transferring the nucleic acid molecule according to the fifth aspect into an immune cell.

Preferably, the transferring is performed via any one of a viral vector, an eukaryotic expression plasmid or mRNA, preferably a viral vector.

Preferably, the CAR-T cell is prepared through transferring the nucleic acid molecule according to the fifth aspect into a T cell via a viral vector.

Preferably, the CAR-T cell is a T cell modified by the viral vector according to the sixth aspect.

In a ninth aspect, the present application provides a composition. The composition includes any one of a combination of at least two of the humanized GD2 scFv according to the first aspect, the derivative antibody conjugate of the humanized GD2 scFv according to the second aspect, the GD2 CAR according to the fourth aspect, the recombinant virus according to the seventh aspect or the CAR-T cell according to the eighth aspect.

In a tenth aspect, the present application provides use of any one of a combination of at least two of the humanized GD2 scFv according to the first aspect, the derivative antibody conjugate of the humanized GD2 scFv according to the second aspect, the GD2 CAR according to the fourth aspect, the recombinant virus according to the seventh aspect, the CAR-T cell according to the eighth aspect or the composition according to the ninth aspect in the preparation of a medicine for treating a tumor.

Preferably, the tumor includes a tumor expressing a GD2-specific antigen.

Preferably, the tumor includes a nervous system tumor expressing the GD2-specific antigen.

Preferably, the tumor includes neuroblastoma.

Compared with the existing art, the present application has the beneficial effects below.

(1) The CAR of the present application has been subjected to specific gene modification on the costimulatory signaling region of the humanized CAR that targets the antigen GD2 on the tumor surface. The modified CAR specifically binds to GD2 and has a better response effect so that the CAR-T cell has a stronger immune response to the tumor, and compared with other GD2 CARs, the CAR has a better long-term effect.

(2) The CAR-T cell of the present application has higher safety and persistence than other GD2 CAR-T cells. Even in a case of a cytokine release syndrome (CRS) caused by an overly strong immune response in a patient, the CAR-T cell can be removed by a drug that induces CAR-T cell apoptosis due to the presence of apoptosis-inducing mechanism. After the CAR-T cell of the present application is infused, a long-term presence of CAR-T can be detected in vivo, which proves that the CAR-T cell has a long-term effect and can achieve an effect of long-term remission of the patient.

(3) A preparation related to the humanized antibody of the present application can play a role in all GD2-positive diseases and has actually been applied to a patient with stage IV neuroblastoma expressing a tumor-specific target GD2. For a patient with minor residues in bone marrow, the preparation has reduced clinical side effects and improved safety and can effectively remove minor residues that are not sensitive to chemotherapy. In addition, GD2 CAR-T is also applied to a patient with glioma in combination with other target CAR-T, and a long-term presence of GD2 CAR-T is detected in the patient, which is conducive to maintaining long-term remission.

### BRIEF DESCRIPTION OF DRAWINGS

FIG. 1 is a schematic diagram of a mechanism of action of a CAR-T cell.

FIG. 2 is a structure diagram of two types of CARs.

FIG. 3 is a plasmid map of a backbone vector pTYF of a lentiviral vector.

FIG. 4A is an image (the magnification is 50×) illustrating that different types of T cells kill GD2-positive tumor cell lines at 24 h and 48 h in vitro.

FIG. 4B is a graph of statistical results of remaining target cells quantified by flow cytometry after different types of T cells kill GD2-positive tumor cell lines at 24 h and 48 h.

FIG. 4C is a graph of statistical results of percentages of dead target cells after different types of T cells kill GD2-positive tumor cell lines at 24 h.

FIG. 5A is a flowchart (the magnification of the inset is 20×) of the treatment of neuroblastoma by using GD2 CAR-T.

FIG. 5B is a curve graph of the detection of CAR copy numbers in vivo after GD2 CAR-T is infused in Example 8.

FIG. 6A is a flowchart (the magnification of the inset is 20×) of a GD2 CAR-T cell in combination with other targets for the treatment of glioma.

FIG. 6B is an image (the magnification is 20×) of immunohistochemical staining results of tumor sections of two patients with glioma.

FIG. 6C is a curve graph of the detection of CAR copy numbers in vivo after GD2 CAR-T is infused in Example 9.

### DETAILED DESCRIPTION

To further elaborate on the technical means adopted and effects achieved in the present application, the present application is further described below in conjunction with examples and drawings. It is to be understood that the specific examples set forth below are intended to explain the present application and not to limit the present application.

Experiments without specific techniques or conditions specified in the examples are conducted according to techniques or conditions described in the literature in the art or a product specification. The reagents or instruments used herein without manufacturers specified are conventional products commercially available from proper channels.

### Example 1

A humanized GD2 scFv was provided. The humanized GD2 scFv had activity of binding to a GD2 antigen.

The humanized GD2 scFv had an amino acid sequence shown in SEQ ID NO. 1.

SEQ ID NO. 1:
QVQLVESGPGVVQPGRSLRISCAVSGFSVTNYGVHWVRQPPGKGLEWLGV
IWAGGITNYNSAFMSRLTISKDNSKNTVYLQMNSLRAEDTAMYYCASRGG
HYGYALDYWGQGTLVTVSSGSTSGSGKPGSSEGSTKGDIVMSQSPSSLAV
SVGEKVTMSCKASQSVSNDVTWYQQKPGQSPKLLIYSASNRYSGVPDRFT
GSGSGTDFTLTISSVKAEDLAVYYCQQDYSSFGAGTKLELK.

### Example 2 Construction of CAR

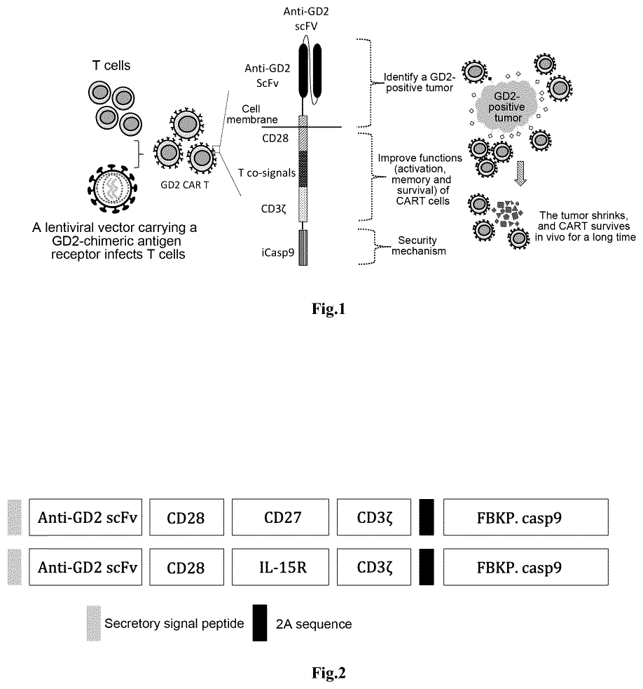

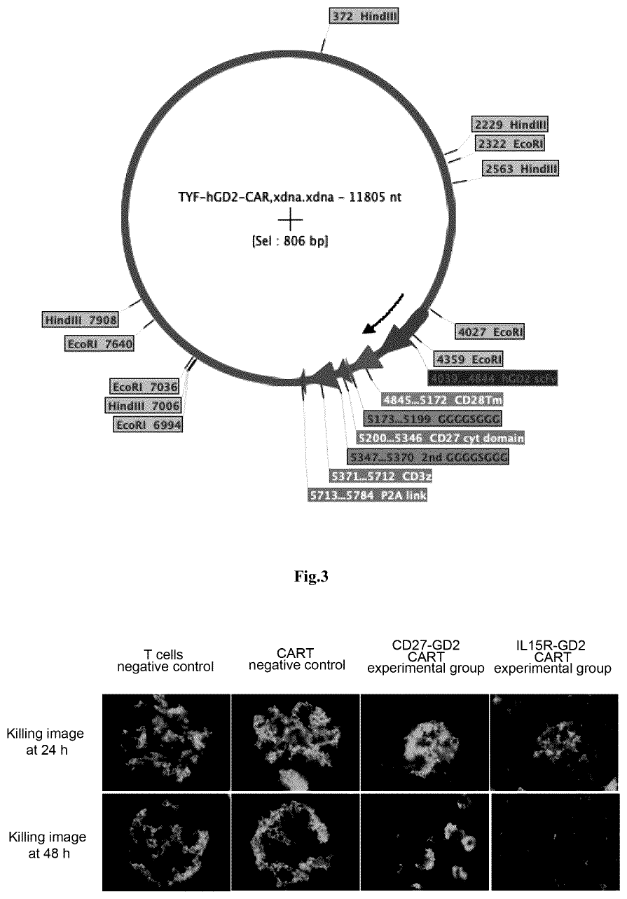

Two types of CARs were provided. The structure diagram of the two types of CARs is shown in FIG. 2.

One CAR was formed by a Secretory signal peptide, a GD2 antigen binding scFv domain, a CD28 transmembrane domain, CD28 and CD27 costimulatory signaling regions, a CD3ζ signaling domain, a 2A sequence and a caspase 9 domain in tandem, and the specific arrangement is as follows: Secretory signal-GD2 scFv-CD28-CD27-CD3ζ-2A-FKBP.Casp9.

The other CAR was formed by a Secretory signal peptide, a GD2 antigen binding scFv domain, a CD28 transmembrane domain, a CD28 and IL-15Ra costimulatory signaling regions, a CD3ζ signaling domain, a 2A sequence and a caspase 9 domain in tandem, and a specific arrangement is as follows: Secretory signal-GD2 scFv-CD28-IL-15Ra-CD3ζ-2A-FKBP.Casp9.

The Secretory signal peptide had an amino acid sequence shown in SEQ ID NO. 6.

SEQ ID NO. 6:
MALPVTALLLPLALLLHAARP.

The CD28 transmembrane domain had a sequence shown in SEQ ID NO. 7.

SEQ ID NO. 7:
FWVLVVVGGVLACYSLLVTVAFIIFWV.

In the construct shown in Secretory signal-GD2 scFv-CD28-CD27-CD3ζ-2A-FKBP.Casp9, “CD28-CD27” included a CD28 hinge region, a CD28 transmembrane domain, a CD28 costimulatory signaling domain and a CD27 costimulatory signaling domain.CD28-CD27 had an amino acid sequence shown in SEQ ID NO. 3.

SEQ ID NO. 3:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSQRRKYRSNKGESPVEPAEPCHYSCPREEEGS
TIPIQEDYRKPEPACSP.

In the construct shown in Secretory signal-GD2 scFv-CD28-IL-15Ra-CD3ζ-2A-FKBP.Casp9, “CD28-IL-15Ra” included a CD28 hinge region, a CD28 transmembrane domain, a CD28 signaling domain and an IL-15Ra signaling domain.CD28-IL-15Ra had an amino acid sequence shown in SEQ ID NO. 4.

SEQ ID NO. 4:
IEVMYPPPYLDNEKSNGTIIHVKGKHLCPSPLFPGPSKPFWVLVVVGGVL
ACYSLLVTVAFIIFWVRSKRSRLLHSDYMNMTPRRPGPTRKHYQPYAPPR
DFAAYRSASGGGGSGGGGSKSRQTPPLASVEMEAMEALPVTWGTSSRDED
LENCSHHL.

The CD3ζ signaling domain had a sequence shown in SEQ ID NO. 8.

SEQ ID NO. 8:
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR.

The 2A sequence had a sequence shown in SEQ ID NO. 9.

SEQ ID NO. 9:
TSGSGATNFSLLKQAGDVEENPGP.

The caspase 9 domain had an amino acid sequence shown in SEQ ID NO. 5.

SEQ ID NO. 5:
MGVQVETISPGDGRTFPKRGQTCVVHYTGMLEDGKKVDSSRDRNKPFKFM
LGKQEVIRGWEEGVAQMSVGQRAKLTISPDYAYGATGHPGIIPPHATLVF
DVELLKLEGGGGSGGGGSGAMVGALESLRGNADLAYILSMEPCGHCLIIN
NVNFCRESGLRTRTGSNIDCEKLRRRFSSLHFMVEVKGDLTAKKMVLALL
ELARQDHGALDCCVVVILSHGCQASHLQFPGAVYGTDGCPVSVEKIVNIF
NGTSCPSLGGKPKLFFIQACGGEQKDHGFEVASTSPEDESPGSNPEPDAT
PFQEGLRTFDQLDAISSLPTPSDIFVSYSTFPGFVSWRDPKSGSWYVETL
DDIFEQWAHSEDLQSLLLRVANAVSVKGIYKQMPGCFNFLRKKLFFKTSA
S.

### Example 3

Two types of lentiviral vectors encoding the two types of CARs in Example 2 were prepared.

The backbone vector of the lentiviral vector was pTYF. For details, see publications such as Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.; Cui, Y. and Chang, L.-J. (2003) Detection and selection of lentiviral vector transduced cells. “Methods in Molecular Biology Vol. 229: Lentivirus Gene Engineering Protocols” pp 69-85, Ed. Maurizio Federico, Humana Press, Inc; Oka, M. Chang, L.-J., Costantini, F., and Terada, N. (2005) Lentivirus mediated gene transfer in embryonic stem cells. Series: “Methods in Molecular Biology” Embryonic Stem Cells 2.

A plasmid map is shown in FIG. 3.

### Example 4 Lentivirus Packaging

Two types of recombinant lentiviruses were prepared. The two types of recombinant lentiviruses were obtained by co-transferring the lentiviral vector in Example 3 and packaging helper plasmids into a mammalian cell. Steps are described below.
(1) 293 T cells were cultured for 17 to 18 h.
(2) Fresh Dulbecco's modified eagle's medium (DMEM) (Thermo Fisher Scientific Inc.) was added.
(3) The following reagents were added to a sterile centrifuge tube: DMEM, packaging helper plasmids (pNHP and pHEF-VSV-G) and a pTYF CAR DNA vector were added to each well (for specific operations, see Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.) and vortexed to be oscillated.
(4) SuperFect (QIAGEN N.V.) was added to the centrifuge tube, and the resulting mixture was left to stand at room temperature for 7 to 10 min.
(5) The DNA-SuperFect mixture in the centrifuge tube was added dropwise to the cultured cells and vortexed.
(6) The cells were cultured in a 37° C. CO₂ incubator for 4 to 5 h.
(7) The culture solution was aspirated, the culture medium was rinsed with AIM-V (BRL), and new AIM-V was added to continue the culture.
(8) The cells were placed back to the CO₂ incubator and cultured overnight. The transduction efficiency was observed the next day.

### Example 5 Purification and Concentration of Lentiviruses

### 1. Purification of Viruses

Cell debris was removed through centrifugation (1000 g, 5 min) to obtain the virus supernatant, the virus supernatant was filtered by a 0.45 μm low protein binding filter, and the viruses were divided and stored at −80° C.

Generally, the transduced cells may produce lentiviral vectors having a titer of 10⁶ to 10⁷ transduction units per milliliter of the culture medium.

2. Concentrating Lentiviruses with Centrifugal Filter
(1) In a biosafety cabinet, a concentrated centrifuge tube was taken, disinfected and aseptically washed twice.
(2) The virus supernatant was added to each centrifugal filter tube and centrifuged until the virus volume was reduced by a factor of 20 to 50.
(3) The filter tubes were shaken, the concentrated viruses were collected through centrifugation into a collection cup, and the viruses in all tubes were collected into one centrifuge tube.

### Example 6 Preparation of CAR-T Cells

Two types of CAR-T cells expressing the CARs in Example 2 were prepared. The preparation method is described below.

The activated T cells were suspended in a culture solution, and 10 μg/mL of polybrene (Sigma) was added to the culture solution. The culture solution was AIM-V containing cell culture factors IL-2, IL-7 and IL-15 (all purchased from PeproTech), and the concentrated lentiviruses in Example 5 were separately added to the culture solution. After centrifugation at 100 g for 100 min at room temperature, the cells were cultured for 24 h at 37° C., and a culture solution was added. After four days of culture, the cells were harvested and counted. After two days of culture, the cells were infused into a patient.

### Example 7 In Vitro Killing Experiment of CAR-T

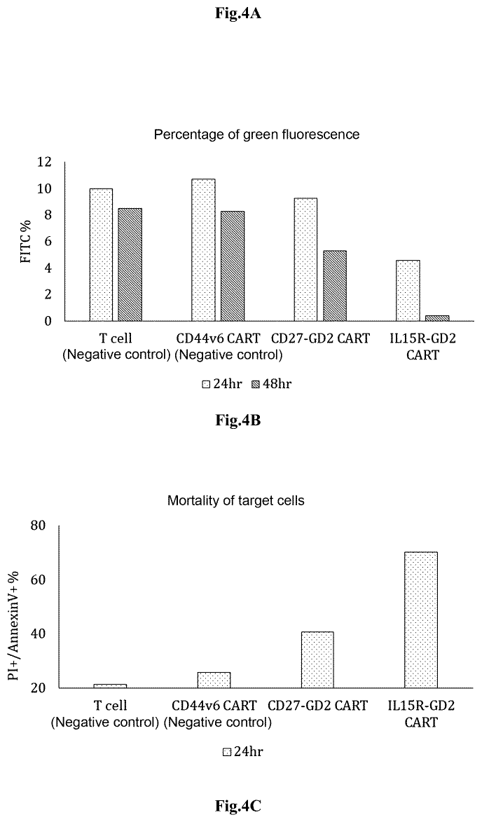

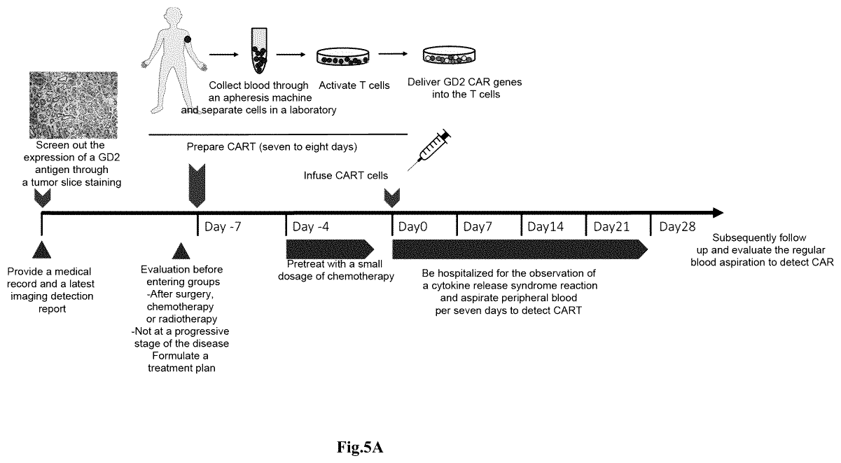

(1) Green fluorescent proteins were transferred into GD2-positive tumor cell lines through lentiviral vectors for stable expression.
(2) T cells and non-specific CAR-T cells (CD44v6 CAR-T) were used as negative control groups, and the two types of CAR-T in Example 6 were used as experimental groups: one structure corresponds to GD2 ScFv-CD28-CD27-CD3ζ, abbreviated as CD27-GD2 CAR-T; the other structure corresponds to GD2 ScFv-CD28-IL15R-CD3ζ, abbreviated as IL15R-GD2 CAR-T. The above four types of cells were co-cultured with the tumor in step (1) in a 5% C02 incubator for 24 to 48 h at 37° C., and the situation of killing tumor cells was observed through a fluorescence microscope. The results are shown in FIG. 4A. No green fluorescent protein was expressed in dead tumor cells. It can be seen that the killing effects of the CD27-GD2 CAR-T group and the IL15R-GD2 CAR-T group are significantly superior to that of the control group and the killing effect of the IL15R-GD2 CAR-T group is the best.

Moreover, the green fluorescence of remaining target cells was quantified by flow cytometry and statistically analyzed. The results are shown in FIG. 4B. It can be seen that the CD27-GD2 CAR-T group and the IL15R-GD2 CAR-T group have less remaining target cells, indicating that corresponding T cells have a relatively strong killing ability.
(3) The apoptosis of the target cells was quantified by using a PI/AnnexinV stain (Sigma), and the situation of CAR-T killing the target cells was observed. The results are shown in FIG. 4C. It can be seen that the target cells in the CD27-GD2 CAR-T group and the IL15R-GD2 CAR-T group have a relatively high mortality, which is consistent with the previous results.

The above experiment was repeated more than three times.

In conjunction with the results of FIGS. 4A, 4B and 4C, it can be seen that compared with the two negative control groups, the two types of GD2 CAR-T cells each have an apparent killing ability and the killing effect of IL15R-GD2 CAR-T is superior to that of CD27-GD2 CAR-T.

### Example 8 GD2 CAR-T Cells for Treatment of Neuroblastoma

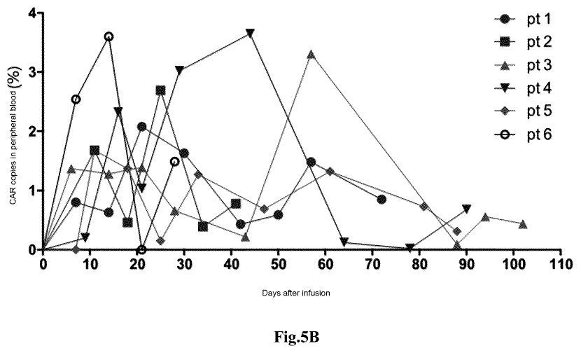

(1) Six patients with stage IV neuroblastoma were recruited as subjects, whose bone marrow cannot be in remission after multi-line treatment, and there were minor residues in bone marrow. A complete treatment flow is shown in FIG. 5A.
(2) The glass slides containing slices of the tumors of the patients were immunohistochemically stained to confirm the positive expression of GD2. Related information is summarized in Table 1.
(3) The concentrate of white blood cells was collected from each patient. Peripheral blood mononuclear lymphocytes in the concentrate of white blood cells were separated through density gradient centrifugation with Ficoll, T cells were screened out by CD3 magnetic beads and activated by an anti-CD28 antibody (BD Biosciences). The subsequent GD2 CAR-T preparation was performed at a rate of 2×10⁶ CAR-T cells/kilogram of the body weight.
(4) Before infused with CAR-T cells, the patients were pretreated with a small dosage of chemotherapy. The pretreatment regimen was cyclophosphamide (250 mg/m²) (Sigma) and fludarabine (25 mg/m²) (Sigma) for three days. CAR-T infusion was conducted 24 h after the pretreatment, which were completed within three days.
(5) CAR-T cells were infused through intravenous injection at dosages shown in Table 1.
(6) After infusion, a clinician monitored the patients and evaluated the toxicity reactions. The clinical toxicity reactions, that is, the CRSs of the six patients after infusion are summarized in Table 1. The results show that no CRS reaction was observed in the six patients.
(7) After infusion, a small amount of peripheral blood was periodically aspirated from each patient, peripheral blood mononuclear lymphocytes were separated, and then cell chromosome DNA (gDNA) was extracted. A CAR copy number in the peripheral blood was quantified through a qPCR (for specific operations, see Chang, L.-J. and Zaiss, A.-K. (2001) Methods for the preparation and use of lentivirus vectors. Methods in Molecular Medicine, Gene Therapy Protocols, 2nd Ed., pp 303-318, Ed. Jeffrey Morgan, Humana Press, Inc.) with a specific primer. The variation curves of CAR copy numbers in the six patients are shown in FIG. 5B, where pt3 is the patient who has been observed for the longest time, and CAR-T copies can still be successfully detected in the peripheral blood after three months.
(8) Before and after GD2 CAR-T was infused, bone marrow aspirates were aspirated by the hospital, and tumor cells in the bone marrow aspirates were detected. Four neuroblastoma patients were cleared of minor residues in bone marrow after infusion, and two patients still had minor residues in bone marrow after infusion. For details, see Table 1.

TABLE 1

| | | | Number of | | Residues | | |
|---|---|---|---|---|---|---|---|
| | | | Effective | | in Bone | | |
| | | GD2 | CART × 10⁶/kg | | Marrow | Residues in | |
| | Gender/ | Expression | at Infusion | CRS | before | Bone Marrow | Clinical |
| No. | Age | Strength | Dosage | Reaction | Infusion | after Infusion | Evaluation |
| N1 | male/6 | 2.5+ | 1.5 | no | 8/990 | three months | complete |
| | | | | | thousand | after infusion, | remission |
| | | | | | cells | negative | |
| N2 | male/8 | 1+ | 2.08 | no | positive | one month after | minor |
| | | | | | | infusion, | residues |
| | | | | | | 0.074% | |
| N3 | male/3 | 2+ | 1.85 | no | 8/3.3 | one month after | complete |
| | | | | | million | infusion, bone | remission |
| | | | | | cells | marrow was | |
| | | | | | | negative | |
| N4 | male/7 | 3+ | 2.41 | no | 22/1.02 | three months | minor |
| | | | | | million | after infusion, | residues |
| | | | | | cells | 5/1.78 million | |
| | | | | | | cells | |
| N5 | female/4 | 1.5+ | 4.4 | no | 9/1.5 | two months | complete |
| | | | | | million | after infusion, | remission |
| | | | | | cells | negative | |
| N6 | male/11 | 2+ | 0.96 | no | positive | one month after | complete |
| | | | | | | infusion, | remission |
| | | | | | | negative | |

### Example 9 GD2 CAR-T Cells in Combination with Other Targets for Treatment of Glioma

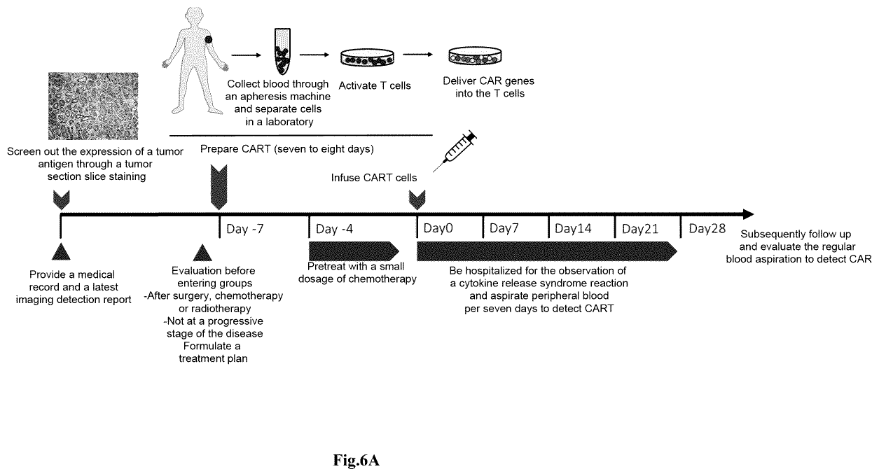

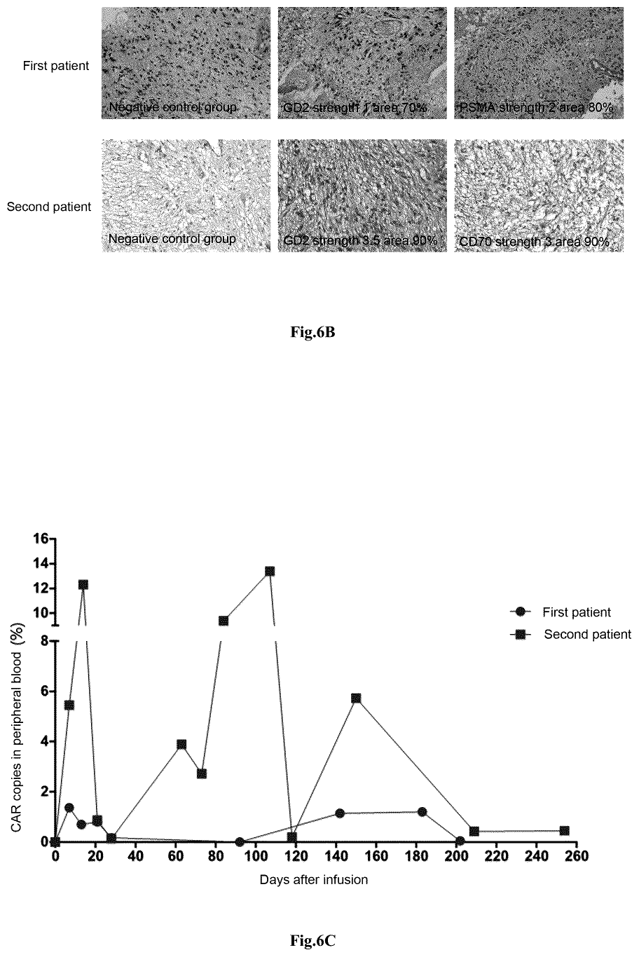

(1) Two patients with refractory glioma were recruited as subjects. A complete treatment flow is shown in FIG. 6A.
(2) The glass slides containing slices of the tumors of the patients were immunohistochemically stained to confirm the expression of targets. Finally, GD2 combined with another tumor antigen with relatively high expression was selected as a CAR-T therapeutic target. The immunohistochemical staining results of the sections are shown in FIG. 6B.
(3) The concentrate of white blood cells was collected from each patient. Peripheral blood mononuclear lymphocytes in the concentrate of white blood cells were separated through density gradient centrifugation with Ficoll, T cells were screened out by CD3 magnetic beads and activated by an anti-CD28 antibody. The subsequent CAR-T preparation was performed at a rate of 2×10⁶ CAR-T cells/kilogram of the body weight.
(4) Before infused with CAR-T cells, the patients were pretreated with a small dosage of chemotherapy. The pretreatment regimen was cyclophosphamide (250 mg/m²) and fludarabine (25 mg/m²) for three days. CAR-T infusion was conducted 24 h after the pretreatment, which were completed within three days.
(5) Two types of CAR-T cells were infused simultaneously through intravenous injection at dosages shown in Table 2.
(6) After infusion, a clinician monitored the patients and evaluated the toxicity reactions. The clinical toxicity reactions, that is, the CRSs of the two patients after infusion are summarized in Table 2. The results show that no CRS reaction was observed in the two patients.
(7) Before and after infusion, tumor lesions of the patients were evaluated through magnetic resonance imaging (MRI). Clinical responses of the patients are summarized in Table 2. One patient was evaluated as stable in the disease, and the other patient achieved an effect of partial remission.
(8) After infusion, a small amount of peripheral blood was periodically aspirated from each patient, peripheral blood mononuclear lymphocytes were separated, and then cell chromosome DNA (gDNA) was extracted. A CAR copy number in the peripheral blood was quantified through a qPCR with a specific primer. The variation curves of CAR copy numbers in the two patients are shown in FIG. 6C. 1.20% and 0.05% CAR-T copies were still in the peripheral blood of the first patient on days 183 and 202 after infusion, and 0.42% and 0.45% CAR-T copies can still be successfully detected in the peripheral blood of the second patient on days 209 and 254 after infusion. It proves that GD2 CAR-T can be maintained in vivo for a long time.

TABLE 2

| | | | | | Detection | | | |
|---|---|---|---|---|---|---|---|---|
| | | | Number of | | of Lesion | Detection | | |
| | | | Effective | | Through | of Lesion | | |
| | | | CART × 10⁶/ | | MRI | Through | | |
| | Gender/ | | kg at Infusion | CRS | before | MRI after | Clinical | |
| No. | Age | Target | Dosage | Reaction | infusion | infusion | Evaluation | Remark |
| 1 | male/61 | GD2 | 2.23 | no | right | right | on day 93 | CART |
| | | PSMA | 3.97 | | parietal | parietal | after | was |
| | | | | | lobe | lobe | infusion, | used in |
| | | | | | 44 × 46 × | 42 × 38 × | the lesion | combination |
| | | | | | 34 cm | 33 cm | shrank | with |
| | | | | | | | without | PD-L1 |
| | | | | | | | achieving a | |
| | | | | | | | standard of | |
| | | | | | | | partial | |
| | | | | | | | remission | |
| 2 | male/3 | GD2 | 4.89 | no | medulla | medulla | on day 38 | no |
| | | CD70 | 15.63 | | oblongata | oblongata | after | |
| | | | | | 30 × 19 | 27 × 17 | infusion, | |
| | | | | | mm | mm | partial | |
| | | | | | 18 × 12 | 12 × 5 | remission | |
| | | | | | mm | mm | was | |
| | | | | | | | achieved | |

In conclusion, the GD2 CAR described in the present application has a better response effect and a better long-term effect. The GD2 CAR is applied to the patient with stage IV neuroblastoma expressing the tumor-specific target GD2. For the patient with minor residues in bone marrow, the GD2 CAR has a smaller clinical side effect and higher safety and can effectively remove minor residues that are not sensitive to chemotherapy. In addition, GD2 CAR-T can also be applied to the treatment for the patient with glioma in combination with other target CAR-T, and the presence of GD2 CAR-T can be successfully monitored in the patient for a long time, which is conducive to maintaining long-term remission.

The applicant has stated that although the detailed method of the present application is described through the examples described above, the present application is not limited to the detailed method described above, which means that the implementation of the present application does not necessarily depend on the detailed method described above. It should be apparent to those skilled in the art that any improvements made to the present application, equivalent replacements of raw materials of the product of the present application, additions of adjuvant ingredients, selections of specific manners, etc., all fall within the protection scope and the disclosure scope of the present application.

1. A humanized disialoganglioside 2 (GD2) single-chain variable fragment (scFv), having activity of binding to a GD2 antigen;
wherein the humanized GD2 scFv has an amino acid sequence having more than 80% identity with SEQ ID NO. 1.

2. A derivative antibody conjugate of the humanized GD2 scFv according to claim 1.

3. A nucleic acid molecule encoding the humanized GD2 scFv according to claim 1; and
preferably, the nucleic acid molecule has a nucleotide sequence having more than 80% identity with SEQ ID NO. 2.

4. A humanized disialoganglioside 2 (GD2) chimeric antigen receptor (CAR), comprising a GD2-antigen-binding single-chain variable fragment (scFv) domain, a transmembrane domain, a costimulatory signaling region, a CD3ζ signaling domain and an inducible suicide fusion domain;
wherein the GD2-antigen-binding scFv domain comprises the humanized GD2 scFv according to claim 1;
preferably, the transmembrane domain comprises a CD28 transmembrane domain and/or a CD8α transmembrane domain;
preferably, the costimulatory signaling region comprises CD28 and CD27 costimulatory signaling regions or CD28 and IL-15Ra costimulatory signaling regions;
preferably, the inducible suicide fusion domain comprises a caspase 9 domain fused to an FK506 binding protein (FKBP);
preferably, the caspase 9 domain fused to the FKBP has an amino acid sequence having more than 90% identity with SEQ ID NO. 5;
preferably, the GD2 CAR further comprises a signal peptide and/or a 2A sequence;
preferably, the signal peptide comprises a Secretory signal peptide; and
preferably, the GD2 CAR comprises a Secretory signal peptide, a GD2-antigen-binding scFv domain, a transmembrane domain, a costimulatory signaling region, a CD3ζ signaling domain, a 2A sequence and an inducible suicide fusion domain.

5. A nucleic acid molecule, encoding the GD2 CAR according to claim 4.

6. A viral vector, comprising at least one copy of the nucleic acid molecule according to claim 5; and
preferably, the viral vector comprises a lentiviral vector or a retroviral vector, preferably the lentiviral vector.

7. A recombinant virus, which is obtained by co-transferring the viral vector according to claim 6 and a packaging helper plasmid into a mammalian cell;
preferably, the packaging helper plasmid comprises pNHP and pHEF-VSVG; and
preferably, the mammalian cell comprises any one of a 293 cell, a 293T cell or a TE671 cell.

8. A chimeric antigen receptor T (CAR-T) cell, expressing the GD2 CAR according to claim 4;
preferably, the transferring is performed via any one of a viral vector, an eukaryotic expression plasmid or mRNA, preferably a viral.

9. A composition, comprising any one or a combination of at least two of the humanized GD2 scFv according to claim 1.

10. (canceled)

11. A method for treating tumor, comprising administering to a patient in need thereof an effective amount of the humanized GD2 scFv according to claim 1;
preferably, the tumor comprises a tumor expressing a GD2-specific antigen;
preferably, the tumor comprises a nervous system tumor expressing a GD2-specific antigen; and
preferably, the tumor comprises neuroblastoma.


SEQUENCE LISTING (11 sequences)

SEQ ID NO: 1 - protein, 241 aa, synthetic construct
QVQLVESGPGVVQPGRSLRISCAVSGFSVTNYGVHWVRQPPGKGLEWLGVIWAGGITNYNSAFMSRLTISKDNSKNTVYLQMNSLRAEDTAMYYCASRGGHYGYALDYWGQGTLVTVSSGSTSGSGKPGSSEGSTKGDIVMSQSPSSLAVSVGEKVTMSCKASQSVSNDVTWYQQKPGQSPKLLIYSASNRYSGVPDRFTGSGSGTDFTLTISSVKAEDLAVYYCQQDYSSFGAGTKLELK

SEQ ID NO: 2 - DNA, 723 bp, synthetic construct
  source 1..723: mol_type = other DNA
caggtccagttggtcgaatctgggcctggggttgtacagccaggtcgaagtcttcgcatatcttgtgcagtatctggcttcagtgttaccaattacggtgtacactgggtgagacaacccccaggtaaagggttggaatggcttggagtgatatgggctgggggcattactaactacaactccgcttttatgtcacggctgactatttcaaaagataactccaagaatacggtgtacttgcaaatgaattcactccgcgcagaggatacagctatgtattattgtgccagtcggggcggccattacggctatgctctggactattggggacaagggacgctcgttacggtatccagtggtagtacgtcaggctccgggaaacctggaagctcagagggtagcaccaaaggcgatatcgtcatgtcacaatccccaagttccctcgcggtaagcgttggggagaaggtaaccatgtcttgtaaggcatctcagtcagtttcaaatgacgtgacgtggtaccaacagaagccgggacaatctcctaagttgttgatctactctgcaagtaacaggtactccggagtgccggaccgctttaccggctctggctcaggcacggacttcacgctgacgatatctagcgtcaaagccgaagacctcgcggtgtattactgtcaacaagattactctagtttcggtgctggaacgaagctggagctgaaa

SEQ ID NO: 3 - protein, 167 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSQRRKYRSNKGESPVEPAEPCHYSCPREEEGSTIPIQEDYRKPEPACSP

SEQ ID NO: 4 - protein, 158 aa, synthetic construct
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSASGGGGSGGGGSKSRQTPPLASVEMEAMEALPVTWGTSSRDEDLENCSHHL

SEQ ID NO: 5 - protein, 401 aa, synthetic construct
MGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLEGGGGSGGGGSGAMVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTSAS

SEQ ID NO: 6 - protein, 21 aa, synthetic construct
MALPVTALLLPLALLLHAARP

SEQ ID NO: 7 - protein, 22 aa, synthetic construct
MLLLVTSLLLCELPHPAFLLIP

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 24 aa, synthetic construct
TSGSGATNFSLLKQAGDVEENPGP

SEQ ID NO: 10 - protein, 5 aa, synthetic construct
GGGGS

SEQ ID NO: 11 - protein, 10 aa, synthetic construct
GGGGSGGGGS